(19) United States
(12) Patent Application Publication
(10) Pub. No.: US 2006/0067963 A1
(43) Pub. Date: Mar. 30, 2006

(54) Gel compositions and methods of producing same

(76) Inventor: Jeffrey Bell, Alpha, NJ (US)

Correspondence Address: REED SMITH LLP, P.O. BOX 488, PITTSBURGH, PA, 15230-0488, US

(21) Appl. No.: 11/211,218

(22) Filed: Aug. 24, 2005

Related U.S. Application Data
(60) Provisional application No. 60/604,328, filed on Aug. 24, 2004.

(51) Int. Cl. A61K 8/81; A01N 25/00

(52) U.S. Cl. 424/401; 424/405

(57) ABSTRACT

Gel compositions and methods of synthesis thereof. The present invention discloses and describes a stable polymeric gel that exhibits highly desirable characteristics, including long term stability over a large temperature range while at the same time including volatile organic compounds into the gel. The gels of the present invention preferably include a volatile hydrophobic liquid, a polymeric material containing a carboxylic acid, and a cross-linking agent. The gel formed using said composition is typically clear although cloudy gels may also be made and the gels made range in firmness from soft to firm. The gels of the present invention may also include volatile organic compounds without compromising the stability of the gel. Such volatile organic compounds may include fragrances, perfumes, fragrance raw materials, deodorants, odor counteractants, malodors, essential oils, ethers, esters, ketones, alcohols, glycols, silicone hydrocarbons, cyclic hydrocarbons, aldehydes, terpines, volatile insecticides, volatile insect repellents, volatile pesticides, volatile antimicrobial agents, volatile fungicides, volatile herbicides and combinations thereof.

The present application claims the benefit under 35 U.S.C. 119(e) of U.S. Provisional Application No. 60/604,328 filed Aug. 24, 2004.

### BACKGROUND OF THE INVENTION

1. Field of the Invention

The present invention relates to the chemical arts. In particular, it relates to a gel composition and methods for producing the same.

2. Description of the Invention

Air freshener gels have been previously described that contain various quantities of volatile hydrophobic materials such as fragrance. Some of those gels are described as clear and some are opaque or translucent.

Clear gel systems have been disclosed in U.S. Pat. Nos. 4,497,663, 4,857,563, 5,780,527, 6,294,162 and WO 01/78792. Those gels are designed to allow the release of volatile hydrophobic materials such as fragrances from the gel structure. The gel systems that have been described previously have used several different compositions. One type of gel is a thermoplastic gel.

Thermoplastic gels exhibit the property of becoming liquid allowing the gel to melt at elevated temperature. That characteristic presents a problem in high temperature applications where a reversible melting gel causes product failure, such as with a car freshener. Poly(ethylene-butylene-styrene) diblock and triblock copolymers and polyamide based gels are examples of thermoplastic gels that exhibit an undesirable melting characteristic for certain applications.

A second type of gel is a thermoset gel. Thermoset gels do not liquefy at elevated temperatures unless the gel degrades. Thermoset gels have sometimes been referred to as elastomeric gels. Previously, gels have been described with compositions based on polyurethanes (WO02/094900), maleinized polymers (U.S. Pat. No. 4,362,841), and polyacrylics (U.S. Pat. No. 6,048,587). Those gels suffer from having very reactive sites that cannot be combined with fragrances for long periods of time without being deactivated. Other gels that use functionalized silicone materials have been described in U.S. Pat. No. 6,075,111 and U.S. application No. 2003/0220425). Those gels suffer from a high cost of raw materials as well as highly reactive active sites. Coatings that utilize a reaction product of an organic acid and a polycarbodiimide have been also disclosed in U.S. application Nos. 2001/0011114 and 2003/0119954.

As discussed above, there has been no incorporation of a volatile hydrophobic liquid into a gel or coating described using polymers containing reactive carboxylic acid groups. Thus, there has been a long-standing need within the field for gels that are stable over a large temperature range and over a long period of time when combined with fragrances or volatile organic compounds.

### SUMMARY OF THE INVENTION

The present invention describes a novel gel composition that has good stability and a wide range of abilities to gel hard-to-gel materials. Additionally, the raw materials of the present invention show long-term stability when combined with fragrances and other potentially reactive hydrophobic liquids. The gels of the present invention preferably include from about 15 to about 95% of a volatile hydrophobic liquid, from about 3 to about 40% of a polymeric compound containing a carboxylic acid, and from about 3 to about 40% of a cross-linking agent, based on the total weight of the polymeric gel composition. In certain presently-preferred embodiments of the present invention, the polymeric compound contains at least about 2.0 carboxylic acid groups per molecule of the polymeric compound.

The present invention also provides methods for the synthesis of such gels. A preferred method of forming the gels of the present invention is to initially mix at least a portion of the volatile hydrophobic liquid with the carboxylated polymer until completely mixed. The cross-linking agent is then preferably added to the mixture and mixed thoroughly and allowed to set at ambient temperature.

### DETAILED DESCRIPTION OF THE INVENTION

The present invention relates to a stable polymeric gel that exhibits highly desirable characteristics including long term stability and over a large temperature range while at the same time including volatile organic compounds into the gel. The gels of the present invention preferably include from about 15 to about 95% of a volatile hydrophobic liquid, from about 3 to about 40% of a polymeric material containing a carboxylic acid moiety, and from about 3 to about 40% of a cross-linking agent, based on the total weight of the polymeric gel composition. The gel formed using said composition is typically clear although cloudy gels may also be made. The gels may range in firmness from soft to firm. The gels of the present invention exhibit minimal syneresis of liquid from the gel.

A preferred method of, forming the gels of the present invention is to initially mix at least a portion the volatile hydrophobic liquid with the carboxylated polymer until completely mixed. The cross-linking agent is then preferably added to the mixture and mixed thoroughly and allowed to set at ambient temperature. The order of addition of the various components of the composition may vary, with the exception that the carboxylated polymer and the cross-linking agent are preferably maintained in separate portions prior to initiating the gellation. The cross-linker may also be mixed with a portion of the volatile hydrophobic liquid prior to being combined with the polymer. The time for the gel to form is temperature dependent. As the temperature is increased, the time for the gel to form is shortened. When the gels of the present invention are made according to the composition described hereinbelow in Example 10, the set time at ambient temperature is 4-24 hours whereas the set time at 180° F. is 18 minutes. The increased rate is proportional to the increased temperature.

Volatile hydrophobic liquids suitable for use in the present invention include fragrances, perfumes, fragrance raw materials, deodorants, odor counteractants, malodors, essential oils, ethers, esters, ketones, alcohols, glycols, silicone hydrocarbons, cyclic hydrocarbons, aldehydes, terpines, volatile insecticides, volatile insect repellants, volatile pesticides, volatile antimicrobial agents, volatile fungicides, volatile herbicides and combinations thereof. The inclusion of volatile or nonvolatile acids is preferably avoided to reduce the potential for cross reaction of the acid with the cross-linker. In a preferred embodiment of the present invention the volatile hydrophobic liquid is a fragrance.

Suitable polymeric materials contain about at least two carboxylic acid moieties on each polymeric molecule. Presently-preferred polymeric materials contain more than two carboxylic acid moieties on each polymeric molecule. Suitable polymeric materials are available from NOVEON (Cleveland, Ohio) and SARTOMER (Philadelphia, Pa.). Preferred polymeric materials may contain a backbone of polybutadiene, polyisoprene, polybutadiene/styrene, or combinations thereof. Suitable molecular weights for these polymers range from about 1000 to about 10,000. Preferred molecular weight range of the polymer is from about 2,000 to about 5,000. One such material is HYCAR CTB 2000×162 from NOVEON. Another suitable material is PRO6340 from SARTOMER.

To form gels of the present invention, the cross-linker are preferably present in the composition at concentration similar to the equivalence of the polymeric material. It is thought that the cross-linker interacts with the carboxylic acid moieties of the polymeric material with that interaction resulting in the covalent binding of the two materials forming a cross-linked matrix. The new structure results in a gel formation that incorporates the hydrophobic liquid within the cross-linked matrix. The preferred equivalence ratio of cross-linker equivalence to the carboxylic acid equivalence within the composition is from about 0.5 to about 2.0. A more preferred equivalence ratio is from about 0.8 to about 1.2.

The cross-linker can be any material that forms a gel when placed in the presence of the carboxylic acid containing polymer. Suitable cross-linkers include polyamides and polycarbodiimides. A preferred polycarbodiimide cross-linker compound is UCARLNK XL-29SE manufactured by DOW CHEMICAL COMPANY, Midland, Mich. Preferred polyamines include diethylenetriamine and ethylene diamine. In addition, combinations of amines may be employed in the present invention.

Other materials may be added to the composition. For example, colorants can be added to the composition including dyes and pigments. A preferred colorant is one or more oil soluble dyes. These dyes are easily added to the hydrophobic liquid prior to inclusion into the composition. Sparkles can also be added to the composition. These can be made of numerous materials including metal or plastic. Other components can be included for aesthetic reasons including natural and artificial botanicals, artificial flowers, seashells, glass, ceramic, sand, paper articles, plastic articles, rocks, and wax. The gel composition of the present invention may be layered above or below other gel compositions. Antioxidants may be added to protect the fragrance and other materials from oxidation. Suitable antioxidants that can be used include BHT and BHA, though other antioxidants are well known to those of skill in the art. UV inhibitors can be added to prevent discoloration. Suitable UV inhibitors include TINUVIN 5060.

Preferably, the gels of the present invention are clear. However, the gels of the present invention may also be opaque. The gel can be made opaque by the addition of opacifying agents. Presently preferred opacifying agents include titanium dioxide, zinc oxide, inorganic salts, waxes, water, and other organic opacifiers that are well known to those of skill in the art.

Additional hydrophobic materials may be added to the gels of the present invention. Such materials may or may not be volatile. These materials include solvents, oils, esters, phthalates, fatty acids, triglycerides, ethers, oils, aliphatic materials, hydrocarbons, plasticizers, and alcohols. Hydrophilic materials may also be added to the gels. Such materials include water, glycols, and alcohols. Additionally, surfactants may be added to the gels of the present invention. Suitable surfactants include nonionic, anionic, cationic, and amphoteric surfactants—examples of which will be well known to those of skill in the art. The inclusion of surfactants may facilitate the inclusion of hydrophilic materials into the gel.

The stability of gels is tested by the following protocol. Gels are stored for four weeks at 50° C., for three days exposed to UV radiation, and for three cycles of freeze/thaw. Those conditions are generally thought to be comparable to one year of shelf life. If gels generally maintain their form, phase, and coloration following such exposure, they are considered stable. In addition, gels described below have been stored for over thirty months with minimal degradation or discoloration.

The examples below demonstrate various embodiments of the present invention.

### EXAMPLE 1

A gel was formed by combining by weight percent 18.7% HYCAR 1300×18 CTBNX, 75% Lemongrass Mint fragrance and 6.3% Dow Hardener #58 (DOW CHEMICAL COMPANY, Midland, Mich.). The mixture was completely blended into a homogeneous mixture and poured into a container. A soft gel formed at room temperature within 24 hours.

### EXAMPLE 2

A gel was formed by combining by weight percent 18.7% HYCAR 1300X18 CTBNX, 75% Lemongrass Mint fragrance and 6.3% UCARLNK XL-295E. The mixture was completely blended into a homogeneous mixture and poured into a container. A soft gel formed at room temperature within 24 hours.

### EXAMPLE 3

A gel was formed by combining by weight percent 18.7% HYCAR 1300X13 CTBN, 75% Lemongrass Mint fragrance and 6.3% UCARLNK XL-295E. The mixture was completely blended into a homogeneous mixture and poured into a container. A soft gel formed at room temperature within 24 hours.

### EXAMPLE 4

A gel was formed by combining by weight percent 18.7% HYCAR 1300×13 CTBN, 75% Lemongrass Mint fragrance and 6.3% UCARLNK XL-295E. The mixture was completely blended into a homogeneous mixture and poured into a container. A soft gel formed at room temperature within 24 hours.

### EXAMPLE 5

A gel was formed by combining by weight percent 15% HYCAR 1300X13CTBN, 75% Lemongrass Mint fragrance and 10% UCARLNK XL-295E. The mixture was completely blended into a homogeneous mixture and poured into a container. A soft gel formed at room temperature within 24 hours.

### EXAMPLE 6

A gel was formed by combining by weight percent 15% HYCAR 2000X162 CTB, 75% Lemongrass Mint fragrance and 10% UCARLNK XL-295E. The mixture was completely blended into a homogeneous mixture and poured into a container. A soft gel formed at room temperature within 24 hours.

### EXAMPLE 7

A gel was formed by combining by weight percent 15% HYCAR 1300X18 CTBNX, 75% Lemongrass Mint fragrance and 10% UCARLNK XL-295E. The mixture was completely blended into a homogeneous mixture and poured into a container. A soft gel formed at room temperature within 24 hours.

### EXAMPLE 8

A gel was formed by combining by weight percent 15% HYCAR 1300×18 CTBNX, 75% Lemongrass Mint fragrance, 5% UCARLNK XL-295E and 5% Dow Epoxy Hardener 58. The mixture was completely blended into a homogeneous mixture and poured into a container. A soft gel formed at room temperature within 24 hours.

### EXAMPLE 9

A gel was formed by combining by weight percent 8% HYCAR 1300X18 CTBNX, 75% Lemongrass Mint fragrance, 5% UCARLNK XL-295E and 5% Dow Epoxy Hardener 58. The mixture was completely blended into a homogeneous mixture and poured into a container. A soft gel formed at room temperature within 24 hours.

Table 1 shows the composition of some additional gels of the present invention.

Table 1. Composition of gel. Values are expressed as weight percent. The crosslinker is UCARLNK XL-295E. The carboxylated polymer is a polybutadiene MW 3200 with about 2.4 equivalents of carboxylation. Each gel was made by dividing the fragrance into two approximately equal portions. The carboxylated polymer was added to one portion of the fragrance and mixed until homogeneous. The crosslinker was added to the second portion of the fragrance and mixed until homogeneous. The two portions were mixed together and poured into a container to gel.

| | | | | | Ex- | Ex- | Ex- |
|---|---|---|---|---|---|---|---|
| | | | | | per. | per. | per. |
| | Exper. | Exper. | Exper. | Exper. | Run | Run | Run |
| Material | Run 1 | Run 2 | Run 3 | Run 4 | 5 | 6 | 7 |
| Citrus | 85 | | | | | 82.9 | |
| Fragrance | | | | | | | |
| Vanilla | | 85 | 85 | | 82.9 | | |
| Fragrance | | | | | | | |
| Suntan | | | | 82.9 | | | |
| Lotion | | | | | | | |
| Fragrance | | | | | | | |
| Pine | | | | | | | 80.1 |
| Carboxylated | 7.5 | 10 | 7.5 | 7.3 | 7.3 | 7.3 | 9.45 |
| Polymer | | | | | | | |
| Crosslinker | 7.5 | 5 | 7.5 | 9.8 | 9.8 | 9.8 | 9.45 |

It will be appreciated by those skilled in the art that the invention may be practiced within a wide range of equivalent parameters, concentrations and conditions without departing from the spirit and scope of the invention and without undue experimentation. While this invention has been described in connection with specific embodiments thereof, it will be understood that it is capable of further modifications. This application is intended to cover any variations, uses or adaptations of the inventions following, in general, the principles of the invention and including such departures from the present disclosure as come within known or customary practice within the art to which the invention pertains.

What is claimed is:

1. A stable gel composition comprising:
from about 15 to about 95% by weight of a volatile hydrophobic liquid;
from about 3 to about 40% by weight of a polymeric compound containing a carboxylic acid moiety; and
from about 1.5 to about 40% by weight of a cross-linking agent.

2. The gel of claim 1, further comprising:
from about 50 to about 85% by weight of said volatile hydrophobic liquid,
from about 5 to about 20% by weight of said polymeric compound containing a carboxylic acid moiety; and
from about 4 to about 20% by weight of said cross-linking agent.

3. The gel of claim 1, wherein said hydrophobic liquid is selected from the group consisting of fragrance, perfume, essential oil, insecticide, insect repellant, pesticide, herbicide, odorant, malodor counteractant, odor masking agent and combinations thereof.

4. The gel of claim 2, wherein said hydrophobic liquid is selected from the group consisting of fragrance, perfume, essential oil, insecticide, insect repellant, pesticide, herbicide, odorant, malodor counteractant, odor masking agent and combinations thereof.

5. The gel of claim 1, wherein said polymeric compound comprises polybutadiene, polybutadiene-styrene, polyisoprene, or combinations thereof.

6. The gel of claim 2, wherein said polymeric compound comprises polybutadiene, polybutadiene-styrene, polyisoprene, or combinations thereof.

7. The gel of claim 1, wherein said polymeric compound contains at least about 2.0 carboxylic acid groups per molecule of said polymeric compound.

8. The gel of claim 2, wherein said polymeric compound contains at least about 2.0 carboxylic acid groups per molecule of said polymeric compound.

9. The gel of claim 1, wherein said cross-linking agent is a polyamine.

10. The gel of claim 9 wherein said polyamine is diethylenetriamine, ethylene diamine, or a combination thereof.

11. The gel of claim 1, wherein said cross-linking agent is a polycarbodiimide.

12. The gel of claim 2, wherein said cross-linking agent is a polycarbodiimide.

13. The gel of claim 11, wherein said cross-linking agent is UCARLNK XL-29SE.

14. The gel of claim 12, wherein said cross-linking agent is UCARLNK XL-29SE.

15. The gel of claim 1, wherein said gel contains a colorant.

16. A method for making a stable polymeric gel composition comprising:
mixing a first mixture, wherein said first mixture comprises:
from about 15 to about 95% by weight of a volatile hydrophobic liquid; and
from about 3 to about 40% by weight of a polymeric compound containing a carboxylic acid, until said first mixture is completely mixed; and
mixing into said first mixture, from about 1.5 to about 40% by weight of a cross-linking agent.

17. A method for making a stable polymeric gel composition comprising:
mixing a first mixture, wherein said first mixture comprises:
from about 15 to about 95% by weight of a volatile hydrophobic liquid; and
from about 3 to about 40% by weight of a polymeric compound containing a carboxylic acid, until said first mixture is completely mixed; and
mixing into said first mixture a second mixture, wherein said second mixture comprises:
from 15 to about 95% by weight volatile hydrophobic liquid; and
from about 1.5 to about 40% by weight of a cross-linking agent.

18. The method of claim 16, wherein the hydrophobic liquid is selected from the group consisting of fragrance, perfume, essential oil, insecticide, insect repellant, pesticide, herbicide, odorant, malodor counteractant, odor masking agent and combinations thereof.

19. The method of claim 17, wherein the hydrophobic liquid is selected from the group consisting of fragrance, perfume, essential oil, insecticide, insect repellant, pesticide, herbicide, odorant, malodor counteractant, odor masking agent and combinations thereof.

20. The gel of claims 16, wherein the polymeric compound is polybutadiene, polybutadiene-styrene, polyisoprene, or combinations thereof.

21. The gel of claims 17, wherein the polymeric compound is polybutadiene, polybutadiene-styrene, polyisoprene, or combinations thereof.

22. The gel of claim 21, wherein said polymeric compound contains at least about 2.0 carboxylic acid groups per molecule of said polymeric compound.
